(19) United States
(12) Patent Application Publication
(10) Pub. No.: US 2021/0395107 A1
(43) Pub. Date: Dec. 23, 2021

(54) PURIFICATION OF AQUEOUS SOLUTIONS CONTAINING FORMALDEHYDE AND USE OF THE PURIFIED SOLUTION IN AN ACRYLIC ACID PRODUCTION PROCESS

(71) Applicant: Arkema France, Colombes (FR)

(72) Inventors: Serge Tretjak, Saint Avold cedex (FR); Michel Fauconet, Saint Avold cedex (FR); Andre Levray, Saint Avold cedex (FR)

(21) Appl. No.: 17/297,565

(22) PCT Filed: Nov. 29, 2019
(86) PCT No.: PCT/FR2019/052840

§ 371 Date: May 27, 2021

(30) Foreign Application Priority Data
Nov. 29, 2018 (FR) FR18.72059

(51) Int. Cl. C02F 1/04; C08F 2/10; C08F 20/06; C07C 45/35; C07C 51/46; C07C 51/48; C07C 51/25; C07C 51/215

(52) U.S. Cl. CPC C02F 1/04; C08F 2/10; C08F 20/06; C07C 45/35; C02F 2101/34; C07C 51/48; C07C 51/252; C07C 51/215; C07C 51/46

(57) ABSTRACT

The present invention relates to a process for treating aqueous effluents containing formaldehyde by distillation in the presence of acetic acid, in particular to a process for treating aqueous solutions resulting from the synthesis of acrylic acid. The invention also relates to the use of the purified aqueous solution in a process for producing acrylic acid by catalytic oxidation of propylene and/or propane in steam dilution.

### TECHNICAL FIELD

The present invention concerns a process for treating aqueous effluents containing formaldehyde, especially a process for treating aqueous solutions from the synthesis of acrylic acid.

The invention also relates to the use of the purified aqueous solution in a process for producing acrylic acid by catalytic oxidation of propylene and/or propane in steam dilution.

### PRIOR ART AND TECHNICAL PROBLEM

Formaldehyde is used as a raw material in the chemical industry, and so generally it is necessary to treat wastewaters containing residual formaldehyde, prior to discharge. Other industrial processes generate formaldehyde as a byproduct, an example being the synthesis of acrylic acid by oxidation of propylene, producing aqueous phases containing formaldehyde which are desirably purified before discharge or in order to recycle them within the process.

There is therefore an ongoing need to effectively treat aqueous effluents containing formaldehyde.

Formaldehyde, also called formic aldehyde, methanal or formol, is a gas at ambient temperature that is highly soluble in water to form hydrates, so making it difficult to separate in the treatment of aqueous effluents comprising formaldehyde as an impurity.

The concentration of formaldehyde in its CH₂O form in an aqueous solution is very low, generally less than 0.1%; the formaldehyde is in the form of methylene glycol, HO(CH₂O)H, and its oligomers HO(CH₂O)ₙH (with n generally from 1 to 8). The formation of polyoxymethylene glycols in the aqueous solution is dependent on temperature and on the presence of other impurities such as acids that are able to catalyze the formation of polymers. These reactions considerably limit the volatility of formol and consequently its separation by distillation, as the vapor pressure of formaldehyde during the distillation is determined by the kinetics of the associated reactions.

To overcome these drawbacks, external compounds are generally employed so as to form adducts with the formaldehyde that are easier to separate from the aqueous medium, by distillation or by absorption on resins.

For example, studies have been conducted by Chen Yu et al, (International Conference on Challenge in Environmental Science and Computer Engineering, 2010) into the removal of formaldehyde after reaction with sodium bisulfite.

A similar example may be found in U.S. Pat. No. 5,545,336, which describes a process for removing formaldehyde with sodium pyrosulfite that has the further advantage of not generating sulfur dioxide in an acidic environment.

In other fields of application, studies have been conducted by Aspi K. Kolah et al. (“Separation Technology” 5 (1995), pp. 13-22), to compare the formaldehyde removal efficacies of different methods in aqueous effluents from the synthesis of 2-butyne-1,4-diol.

These methods are relatively complex to implement and require the introduction of an external compound, which may be detrimental if the purified effluent is to be recycled within a process.

The inventors have found that the presence of acetic acid in an aqueous solution containing formaldehyde makes it easier to separate the formaldehyde from the aqueous solution and allows it to be removed by simple distillation.

The invention accordingly provides a new process for treating aqueous effluents containing formaldehyde by distillation in the presence of acetic acid.

Document FR 2152849 describes a process for extracting acetic acid from a mixture comprising 0.5-10% of formaldehyde and 0.5-15% of water, with the balance being acetic acid. The process involves an extractive distillation with water as stripping agent, by producing a reactive distillation with column-top injection of water to selectively scavenge the formaldehyde and to enable the recovery of pure acetic acid at the column bottom (see example 1 and the process figure). In this case, the water manages to selectively separate the formaldehyde from the acetic acid in spite of a very high level of the acid in the mixture. In the comparative examples of this document, even without addition of water, when a mixture essentially consisting of acetic acid is distilled, formaldehyde is virtually absent (0.1% to 0.2%) from the bottom stream containing more than 99% of acetic acid. This shows that the liquid-vapor equilibria of the formaldehyde in the (bottom) acetic acid mixture are completely different from those of the formaldehyde in the water mixture. In the acetic acid solvent, the formaldehyde is in monomeric form, so giving it a high relative volatility in relation to the acetic acid, and hence explaining why its concentration is very low at the bottom of the distillation column, in the comparative examples as well.

The invention is particularly advantageous for treating aqueous phases generated in a process for synthesizing acrylic acid. The reason is that the synthesis of acrylic acid by catalytic gas-phase oxidation of propylene and/or propane generates water and forms condensable light byproducts, particularly formaldehyde and acetic acid.

The complexity of the gaseous mixture obtained in this process means that a set of operations is needed to recover the acrylic acid and convert it into a purified acrylic acid grade compatible with its eventual use.

Accordingly, the aqueous streams from the acrylic acid purification steps may contain formaldehyde and/or acetic acid.

In the manufacture of acrylic acid by catalytic gas-phase oxidation of propylene and/or propane, the reagent is introduced in the diluted gas-phase state, generally at a volume concentration of 4% to 15%. In general, a portion of the diluting gas is supplied by the nitrogen accompanying the oxygen introduced in the form of air, and the rest is constituted either by the partial recycling of a mixture of inert compounds and residual light products from the step in which the acrylic acid reaction stream is condensed, or by the water vapor originating advantageously from an aqueous stream obtained downstream of the process.

In an acrylic acid manufacturing process using water vapor as a gaseous diluent for the propylene and/or propane, a recycled aqueous stream is generally used that originates from recovery and purification steps in the process, so as to limit consumption of external water.

When the aqueous stream recycled as a steam source contains formaldehyde, it has been found that the formaldehyde acts as a poison of the catalytic reaction. The consequences are a drop in selectivity of the reaction and a decrease in the lifetime of the catalyst. As an example, the recycling of an aqueous stream containing 2% of formaldehyde in the reaction section produces a drop of 1 to 2% in yield of acrylic acid for a given reaction temperature, or an increase of 6 to 7° C. in the reaction temperature to retain the same degree of conversion of the propylene and/or propane. In both cases, moreover, a decrease is observed in the selectivity of the reaction, with more byproducts formed. An increase in the reaction temperature also gives rise to a decrease in the lifetime of the catalyst, which has to be replaced prematurely, resulting in substantial costs.

There is therefore a need for a process for treating aqueous phases from the synthesis of acrylic acid by catalytic oxidation of propylene and/or propane whose efficacy is such as to produce water that is sufficiently pure, i.e., is essentially devoid of formaldehyde, allowing it to be recycled, i.e., reused in the reaction section of a process for synthesizing acrylic acid by catalytic oxidation of propylene and/or propane in steam dilution.

One objective of the present invention is to propose a simple technical solution for removing formaldehyde in order to meet this need and to improve the productivity and the lifetime of the propylene and/or propane oxidation catalyst.

### SUMMARY OF THE INVENTION

A subject of the invention is a process for removing formaldehyde by distillation from an aqueous solution containing formaldehyde, characterized in that the distillation is performed in the presence of acetic acid.

In one embodiment, the aqueous solution contains from 0.1 to 5% by mass, preferably from 1 to 3% by mass of formaldehyde. In one embodiment, the aqueous solution contains from 1 to 10% by mass, preferably from 2 to 6% by mass of acetic acid.

Advantageously, the mass ratio of the acetic acid to the formaldehyde in the aqueous solution is between 1 and 5, preferably between 1 and 4.

In one embodiment, the distillation is carried out using a distillation column fitted with a top-mounted top condenser.

In one embodiment, the distillation is carried out using a distillation column fitted with a top-mounted mechanical vapor compressor.

In one embodiment, the aqueous solution containing formaldehyde is from a process for synthesizing acrylic acid by catalytic oxidation of propylene and/or propane.

In this embodiment, the acetic acid may be in the aqueous solution undergoing the treatment, or the acetic acid is added by way of a stream comprising acetic acid generated in said process for synthesizing acrylic acid.

In one embodiment, the process for synthesizing acrylic acid includes a process for purifying acrylic acid comprising water separation by liquid extraction using a solvent.

In one embodiment, the process for synthesizing acrylic acid includes a process for purifying acrylic acid comprising water separation by azeotropic distillation using a solvent.

In one embodiment, the process for synthesizing acrylic acid is a process for catalytic oxidation of propylene and/or propane in steam dilution, in other words being fed with a stream of starting materials diluted in water vapor.

In one embodiment, the aqueous phase after treatment is recycled to the process for synthesizing acrylic acid, preferably as a steam source in the reaction section.

Another subject of the invention is a process for synthesizing acrylic acid by catalytic oxidation of propylene and/or propane, comprising the distillation treatment of an aqueous phase containing formaldehyde and acetic acid, in an acetic acid/formaldehyde mass ratio of from 1 to 4, and the recycling of the purified aqueous phase as a steam source in the reaction section of the process, said aqueous phase being recovered at the bottom of the distillation column, while the formaldehyde is recovered at the column top.

In one embodiment, the distillation treatment is carried out using a dividing-wall distillation column, allowing separate removal of the formaldehyde and of the residual solvents dissolved in the aqueous phase, which can be recycled.

The inventors have surprisingly found that the presence jointly of acetic acid and formaldehyde in the aqueous stream feeding a distillation column makes it possible to remove a greater part of the formaldehyde present, at the top of the distillation column.

The treatment process according to the invention is therefore particularly advantageous for removing the formaldehyde present in aqueous streams containing acetic acid that are generated in an acrylic acid synthesis process; the acetic acid may be present directly in the stream for treatment, owing to favored entrainment thereof under certain operating conditions in the acrylic acid purification procedure. As an alternative, the acetic acid is added by way of a concentrated stream of acetic acid produced within the acrylic acid purification process. These two alternatives have the advantage of not introducing phases or external products that can pollute the stream for treatment, and they result in an aqueous phase which is essentially devoid of formaldehyde and is suitable for use as a source of steam for diluting the gases entering the reaction section of the acrylic acid synthesis process. The result of this is a gain in terms of water consumption within the process.

The energy balance of the acrylic acid synthesis process can also be optimized by combining mechanical recompression of the steam distilled at the top of the distillation column, allowing this steam to be used as a heat transfer fluid.

### BRIEF DESCRIPTION OF THE FIGURES

FIG. 1 represents schematically an acrylic acid production plant with water separation by liquid extraction and inventive purification of the aqueous stream recycled as a steam source.

FIG. 2 represents schematically an acrylic acid production plant with water separation by azeotropic distillation and inventive purification of the aqueous stream recycled as a steam source.

FIG. 3 represents a variant of the inventive purification process which can be used in the plant of FIG. 1 and of FIG. 2.

FIG. 4 illustrates the effect of the presence of acetic acid on the removal of formaldehyde by distillation.

### DETAILED ACCOUNT OF THE INVENTION

The invention is now described in greater detail and in a nonlimiting manner in the description which follows.

The treatment process according to the invention is performed by distillation using a conventional distillation column which may comprise at least one packing element, for instance bulk packing and/or structured packing, and/or trays, for instance perforated trays, fixed valve trays, movable valve trays, bubble trays, or combinations thereof.

The distillation column preferably comprises a number of theoretical plates of between 1 and 15 and operates at atmospheric pressure.

In one embodiment, the distillation column is fitted with a top-mounted top condenser which condenses the vapors that are generated. The condensed product can be at least partly recycled as reflux in the distillation column, with the remainder being advantageously withdrawn and recycled in whole or in part in the process: for example, in a step of absorbing acrylic acid in the gaseous mixture from the reaction section, or sent to a treatment station for later treatment before discharge.

In one embodiment, the distillation column is fitted with a top-mounted mechanical vapor compressor, bringing the vapour to a pressure such that the temperature attained is greater than the temperature of the column bottom. The vapors compressed in this way may be used as a heat transfer fluid supplying a part of the heat flow needed at the boiler associated with the distillation column, to provide for the distillation.

The aqueous solution treated by distillation according to the invention contains in general from 0.1 to 5% by mass of formaldehyde.

An amount of from 1 to 10% by mass of acetic acid in the aqueous solution promotes the removal of the formaldehyde by distillation.

In general, a mass ratio of acetic acid to formaldehyde of between 1 and 4 enables a degree of removal of the formaldehyde of greater than 60%, or even greater than 70%.

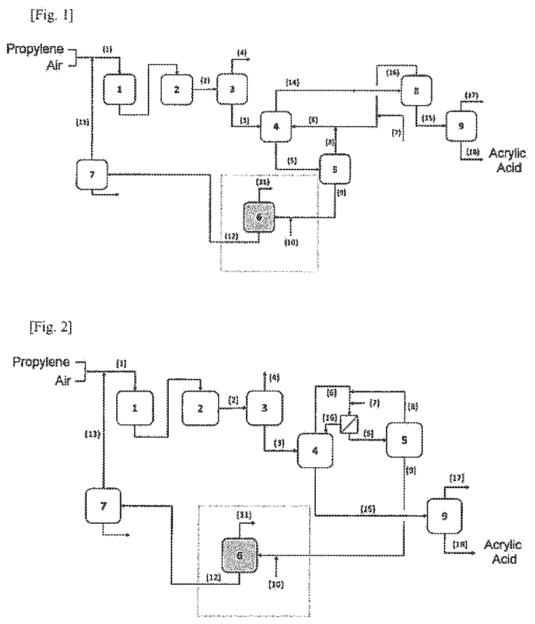

In one preferred embodiment, the aqueous solution submitted to the process according to the invention is generated by an acrylic acid purification procedure employed in a process for producing acrylic acid by catalytic gas-phase oxidation of propylene and/or propane. One such aqueous solution is represented, for example, by the stream (9) in FIGS. 1 and 2.

Referring to FIGS. 1 and 2, a plant for producing acrylic acid comprises a first reactor 1 fed with a mixture (1) of propylene and/or propane and oxygen and in which a mixture rich in acrolein is produced, which is sent to a second reactor 2, where the acrolein is selectively oxidized to acrylic acid.

The gaseous mixture (2) from the second step consists—as well as of acrylic acid

of unconverted compounds from the reactants employed or of impurities generated during one or both of the reaction steps, these constituents being

light compounds which are incondensable under the temperature and pressure conditions normally used: essentially propylene, propane, nitrogen, unconverted oxygen, carbon monoxide and dioxide formed in small amounts by final oxidation;

light compounds which are condensable: essentially water, light aldehydes such as unconverted acrolein, formaldehyde and acetaldehyde, formic acid, acetic acid, propionic acid;

heavy compounds: especially furfuraldehyde, benzaldehyde, maleic acid and anhydride, benzoic acid.

The complexity of the gaseous mixture (2) obtained in this process means that a set of operations is needed to recover the acrylic acid present in this gaseous effluent and convert it into an acrylic acid grade compatible with its eventual use.

For this purpose, the gaseous mixture (2) is sent to an absorption column 3, where the acrylic acid and other oxidation products are condensed by absorption with water, and a stream (4) of incondensable compounds is removed.

The liquid stream (3) leaving the absorption column 3 undergoes a dehydration step, which is carried out in the presence of a water-immiscible solvent (7) for the acrylic acid, in a unit 4.

In a first variant, shown in FIG. 1, the dehydration step is carried out by liquid-liquid extraction of the acrylic acid in the presence of the solvent (7) in a liquid extraction column 4, generating a bottom stream (5) containing water and impurities including formaldehyde, and a top stream (14) rich in acrylic acid in solvent medium. Solvents which can be used include, for example, ethyl acrylate or isopropyl acetate.

The stream (14) then undergoes a distillation 8 to recover the solvent (16), which is recycled by way of the stream (6) into the extraction column 4, with the bottom stream (15) undergoing purification in a distillation column 9, producing at the bottom a technical-grade acrylic acid (18), and at the top a stream concentrated with light impurities.

In a second variant, shown in FIG. 2, the dehydration step is carried out by azeotropic distillation with a solvent (7) in a distillation column 4, producing a two-phase medium (6) at the column top: an organic phase (16) consisting essentially of the solvent, which is recycled in reflux in the column 4, and an aqueous phase (5) containing impurities including formaldehyde. Solvents which can be used include, for example, methyl isobutyl ketone (MILK) or toluene.

At the bottom of the azeotropic distillation column, the stream (15) undergoes purification in a distillation column 9, producing at the bottom a technical-grade acrylic acid (18), and at the top a stream (17) concentrated with light impurities.

Other steps, not shown in FIGS. 1 and 2, may be present in the acrylic acid purification section.

In these two variants, the aqueous stream (5), containing a small amount of dissolved solvent, is advantageously sent to a step for solvent recovery by distillation in a column 5; the solvent is recovered at the top (8) and recycled into the stream (6) feeding the unit 4, and an aqueous phase containing essentially all the formaldehyde is obtained at the bottom (9).

The process according to the invention involves treating the aqueous phase (9) by distillation in a distillation column 6, so as to remove essentially all of the formaldehyde present, in the top stream (11), and obtain a purified aqueous phase (12).

The invention involves performing the distillation in the column 6 in the presence of acetic acid, either by adding this compound by way of a stream (10), external or generated within the process, preferably by way of a recycled stream, or by performing the acrylic acid purification/recovery process in such a way as to promote the entrainment of the acetic acid impurity into the stream (9). The distillation is preferably carried out at atmospheric pressure in the column 6, said stream 9 being introduced at the bottom third of this column.

One advantageous way of introducing the acetic acid into the stream (9) before distillation of the formaldehyde is to use a stream (10) in the form of a stream concentrated with acetic acid, obtained at the top of a column for separating this impurity.

In FIGS. 1 and 2, a stream of this kind concentrated with acetic acid is represented by the stream (17), which is obtained during the distillation of the acrylic acid (18) recovered at the bottom of the distillation column 9.

The aqueous phase (12), from which essentially all the formaldehyde has been removed, is advantageously sent to a steam generator 7, and the water vapor generated (13) is sent to the reaction section of the process, for diluting the propylene/propane at the entry of the first reactor, and producing a volume concentration of propylene/propane of between 5% and 10% in the reactor 1.

The stream (11) distilled at the top of the column 6, containing the formaldehyde, can be removed, or at least partly recycled in the process.

In one embodiment, the stream (11) is recycled at the top of the acrylic acid absorption column 3. The formaldehyde is then entrained with the inert gases and the uncondensed light compounds into the column-top stream (4), which stream (4) can be removed by incineration.

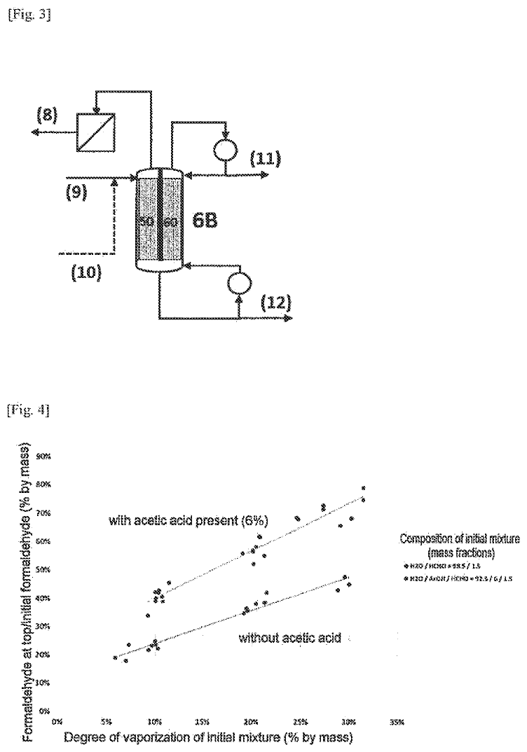

According to the invention, a third variant involves combining the step of recovering the solvent present in the formaldehyde-containing aqueous phase with the removal of the formaldehyde by distillation in the presence of acetic acid. These two steps are combined using a single dividing-wall distillation column as shown for example in FIG. 3.

A dividing-wall column 6B is fed directly with an aqueous phase (9) containing formaldehyde and a small amount of dissolved solvent, obtained from the step of dehydrating the reaction mixture. A stream (10) comprising acetic acid may be added under the conditions described above.

Column 6B fulfills the same function as the distillation columns 5 and 6 positioned in series in the schemes shown in FIGS. 1 and 2.

The following is a possible configuration of the column 60: The distillation column 6B comprises a dividing wall joined to the upper dome of the column at the top part and not joined to the base of the column at the bottom part, the wall thus separating the column into two sections, the lower space thereof communicating with the column-base space, and the top space thereof being separated into two hermetic zones.

The column 6B is supplied at the top plate of the feed section 50. At the top of the section 50 a top stream (8) comprising the solvent is distilled and can be recycled.

At the section 60 called the withdrawal section, a formaldehyde-rich stream (11) is distilled at the top and a stream (12) corresponding to the aqueous phase from which essentially all of the formaldehyde has been removed is recovered at the bottom, and this stream (12) can advantageously be recycled as a steam source.

A further subject of the invention is a process for synthesizing acrylic acid by is catalytic oxidation of propylene and/or propane, comprising at least one step generating an aqueous phase containing from 0.1 to 5% of formaldehyde, in which the formaldehyde in said aqueous phase is removed at the top of a distillation column, characterized in that the distillation is performed in the presence of acetic acid, in an acetic acid/formaldehyde mass ratio of from 1 to 5, the purified aqueous phase obtained at the column bottom being recycled as a steam source in the reaction section of the process.

The following examples illustrate the present invention and are not aimed at limiting the scope of the invention as defined by the appended claims.

### Experimental Section

An assembly was used comprising a 200 mm-diameter distillation column comprising 5 m of Pall rings, equivalent to 10 theoretical plates.

The column was fed at a point situated in the lower part (third) with an aqueous stream comprising formaldehyde, which underwent distillation at atmospheric pressure five degrees below the bubble point of the feed plate. The column is fitted with a top-mounted pin-type condenser. The gaseous phase is sent to a vent and the liquid phase is withdrawn and passed into a tray which is placed on a balance. The reflux rate is provided by the positioning time of an automatic 3-way valve to the reflux of the column or to the withdrawal line.

Distillations were carried out with variation of:
the reflux rate, expressed by the liquid flow returned to the column relative to the flow withdrawn at the column top, between 0.5 and 5, and/or
the degree of distillation, expressed by the mass percentage between the flow withdrawn at the column top and the feed flow to the column, between 10 and 30%.

For the different experiments conducted, a mass balance in relation to the amount of formaldehyde present in the feed stream and in the distilled stream was performed by high-performance liquid chromatography after complexation with dinitrophenylhydrazine.

This enabled determination of a degree of separation of the formaldehyde, expressed by the mass percentage between the flow of formaldehyde at the column top and the feed flow of formaldehyde.

Two aqueous streams comprising formaldehyde were tested:
water comprising 1.5% by mass of formaldehyde (comparative)
water comprising 1.5% by mass of formaldehyde and 6% by mass of acetic acid (inventive).

The results are collated in FIG. 4, which shows the degree of separation of formaldehyde as a function of the degree of distillation, for the two streams tested.

The degree of removal of the formaldehyde remains lower than 50% in the absence of acetic acid, thus confirming the difficulty of distilling formaldehyde.

The presence of acetic acid allows the degree of removal of the formaldehyde to be taken to more than 70%.

1. A process for removing formaldehyde by distillation from an aqueous solution containing formaldehyde wherein the distillation is performed in the presence of acetic acid.

2. The process as claimed in claim 1, wherein the aqueous solution contains from 0.1 to 5% by mass of formaldehyde.

3. The process as claimed in claim 1 wherein the aqueous solution contains from 1% to 10% by mass of acetic acid.

4. The process as claimed in claim 1 wherein the mass ratio of the acetic acid to the formaldehyde in the aqueous solution is between 1 and 5.

5. The process as claimed in claim 1 wherein the distillation is carried out using a distillation column fitted with a top-mounted top condenser.

6. The process as claimed in claim 1 wherein the distillation is carried out using a distillation column fitted with a top-mounted mechanical vapor compressor.

7. The process as claimed in claim 1 wherein the aqueous solution containing formaldehyde is from a process for synthesizing acrylic acid by catalytic oxidation of propylene and/or propane.

8. The process as claimed in claim 7, wherein the acetic acid is added by way of a stream comprising acetic acid generated in said process for synthesizing acrylic acid.

9. The process as claimed in claim 7 wherein said process for synthesizing acrylic acid includes a process for purifying acrylic acid comprising water separation by liquid extraction using a solvent.

10. The process as claimed in claim 7 wherein said process for synthesizing acrylic acid includes a process for purifying acrylic acid comprising water separation by azeotropic distillation using a solvent.

11. The process as claimed in claim 1 wherein the aqueous phase after treatment is recycled to said process for synthesizing acrylic acid, said aqueous phase being recovered at the bottom of the distillation column, while the formaldehyde is recovered at the column top.

12. A process for synthesizing acrylic acid by catalytic oxidation of propylene and/or propane, comprising at least one step: generating an aqueous phase containing from 0.1 to 5% of formaldehyde, in which the formaldehyde in said aqueous phase is removed at the top of a distillation column, wherein the distillation is performed in the presence of acetic acid, in an acetic acid/formaldehyde mass ratio of from 1 to 5, the purified aqueous phase obtained at the column bottom being recycled as a steam source in the reaction section of the process.

13. The process as claimed in claim 12, wherein the distillation treatment is carried out using a dividing-wall distillation column.
